(19) United States
(12) Patent Application Publication
(10) Pub. No.: US 2005/0281839 A1
(43) Pub. Date: Dec. 22, 2005

(54) Spirulina composition and antiallergic food

(76) Inventors: Amha Belay, La Quinta, CA (US); M. Eric Gershwin, Davis, CA (US)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/869,928

(22) Filed: Jun. 18, 2004

(51) Int. Cl. A61K 35/80; A61K 33/32; A61K 31/555; A61K 31/315

(52) U.S. Cl. 424/195.17; 424/641; 514/184; 514/494

(57) ABSTRACT

The present invention provides a Spirulina composition capable of inhibiting the production of IL-4, and to an antiallergic food comprising the Spirulina composition, and also provides a method for prevention or improvement of allergic diseases, which includes ingestion of a composition comprising the Spirulina composition. The Spirulina composition contains Spirulina and a zinc compound, and the content of the zinc compound is from 0.04 to 5 parts by weight, in terms of zinc, based on 100 parts by weight of the dry weight of Spirulina. Ingestion of the Spirulina composition inhibits the production of IL-4, thus exerting the effect of preventing or improving allergic diseases. Also various foods comprising the Spirulina composition can be used as an antiallergic food because the effect of preventing or improving allergic diseases can be expected.

### BACKGROUND OF THE INVENTION

1. Field of the Invention

The present invention relates to a Spirulina composition capable of inhibiting the production of interleukin-4 (IL-4), a method including ingestion of the Spirulina composition, and an antiallergic food comprising the Spirulina composition.

2. Description of Related Art

Recently, various allergosis such as grass allergy, atopic dermatitis and bronchial asthma have become serious problems. Steroid-based medicaments having high pharmacological effects are generally used for allergic diseases, but exert various side effects due to the inhibition of adrenal gland function and inhibition of the growth of skin cells. Also non-steroid-based medicaments such as agents capable of releasing histamine or leukotriene and agents capable of inhibiting the production of an immune globulin E (IgE) antibody are used, but exert insufficient effects.

It has been known that IL-4 is glycoprotein which is produced by T lymphocytes stimulated by an antigen, and also enhances the production of an IgE antibody associated with allergic diseases by an action on B lymphocytes. Therefore, it is believed that allergic diseases can be prevented or improved when the production of IL-4 can be inhibited.

Additionally, there are various restrictions on the above-described medicaments, and for example, the medicaments are generally expensive and require a prescription when obtained, and also require that attention be paid to side effects. Therefore, it is required to develop a food (functional food) which is free of the restrictions of the medicaments, for example, a food which is easily available and is less likely to cause any side effects, and is also simply ingestible, in order to prevent or improve allergic diseases.

As such a functional food, for example, Spirulina is known. Spirulina is a prokaryote which belongs to the genus Arthrospira or Spirulina of the blue-green algae and grows naturally in Lake Chad in Africa. Spirulina is rich in proteins, vitamins, lipids, glucides and vegetable pigments and has high rate of digestion and absorption, and is also formulated into preparations by a compression or capsuling technique or is used in the form of a dry powder. Also it is known that Spirulina contains minerals such as calcium (400 to 1200 mg: the content in 100 g of Spirulina, this is repeated in the following), potassium (800 to 2000 mg), iron (50 to 150 mg), zinc (1 to 3 mg), and copper (0.3 to 0.6 mg) (written by Ikuo Saiki, “Extremely Complete Food Spirulina”, issued by Takanawa Shuppansha, Dec. 20, 1996, pp. 62-65).

Various studies involving the function of Spirulina have been made. For example, the present inventors have confirmed that an effect of phosphate buffered saline extract of Spirulina on peripheral blood mononuclear cells collected from healthy human subjects causes a remarkable increase in production amount of interferon-γ (IFN-γ) capable of inhibiting the growth of tumor cells, as compared with a controls, and reported that Spirulina promotes the production of IFN-γ. However, the production amount of IL-4 did not decrease (M. E. GERSHWIN, et. al., Effect of Spirulina on the Secretion of Cytokines from Peripheral Blood Mononuclear Cells, Journal of Medical Food, 2000, Vol. 3, p. 135-140).

### SUMMARY OF THE INVENTION

An object of the present invention is to provide a Spirulina composition capable of inhibiting the production of IL-4, and an antiallergic food comprising the Spirulina composition.

To achieve the above object, the present inventors have intensively researched and obtained the following findings (1) and (2):
(1) Spirulina containing a trace amount of a zinc compound (for example, 1 to 3 mg in terms of zinc) as a mineral ingredient exert no IL-4 production inhibitory effect, as reported by M. E. GERSHWIN, et. al., while a Spirulina composition containing a larger amount of a zinc compound (for example, 0.04 to 5 parts by weight, in terms of zinc, based on 100 parts by weight of the dry weight of Spirulina) can effectively inhibit the production of IL-4; and
(2) a food containing the Spirulina composition is suited for use as an antiallergic food for prevention or improvement of allergic diseases.

The present invention has been completed based on the findings described above.

The present invention provides a Spirulina composition comprising Spirulina and a zinc compound, wherein the content of the zinc compound is from 0.04 to 5 parts by weight, in terms of zinc, based on 100 parts by weight of the dry weight of Spirulina.

Also the present invention provides an antiallergic food comprising the Spirulina composition.

Ingestion of the Spirulina composition of the present invention inhibits the production of IL-4, thus exerting the effect of preventing or improving allergic diseases. Also various foods comprising the Spirulina composition can be used as an antiallergic food because the effect of preventing or improving allergic diseases can be expected.

### DETAILED DESCRIPTION OF THE INVENTION

The present invention will be described in more detail below.

It is indispensable that the Spirulina composition of the present invention contain Spirulina and a zinc compound and that the content of the zinc compound be from 0.04 to 5 parts by weight, in terms of zinc, based on 100 parts by weight of the dry weight of Spirulina. A content of the zinc compound of less than 0.04 parts by weight is not preferable because less IL-4 production inhibitory effect is exerted. Also, a content of the zinc compound of more than 5 parts by weight is not preferable because an improved IL-4 production inhibitory effect cannot be expected and ingestion of excess zinc may injure one's health. The content of the zinc compound is preferably from 0.07 to 0.5 parts by weight, and more preferably from 0.1 to 0.3 parts by weight, in terms of zinc, based on 100 parts by weight of the dry weight of Spirulina.

Examples of Spirulina used in the present invention include Arthrospira (Spirulina) platensis, Arthrospira (Spirulina) maxima, Spirulina subsalsa, Spirulina major, Spirulina geitleri, Spirulina siamese, Spirulina princeps, Spirulina laxissima, Spirulina curta, and Spirulina spirulinoides. Among these, Arthrospira (Spirulina) platensis, Arthrospira (Spirulina) maxima, Spirulina geitleri and Spirulina siamese are preferable because they can be artificially cultivated and therefore easily available.

Spirulina used in the present invention may be a commercially available one or an artificially cultivated one. Also fresh Spirulina or dry Spirulina may be used, and fresh Spirulina is preferable because fresh Spirulina is enriched in nutrients of Spirulina.

Spirulina may be cultivated by a conventional method used in the cultivation of blue-green algae and, for example, viable Spirulina is usually inoculated in a culture medium in a concentration of 20 to 500 mg/l based on the weight of the algal dry biomass, and then cultivated. A light source used in the cultivation may be sunlight or artificial light, but is usually a light source of 1,000 to 100,000 lux. The cultivation temperature is usually from 20 to 65° C., and preferably from 30 to 40° C. The cultivation period is usually from 5 to 10 days. During the cultivation, the amount of carbonate ions in the solution is preferably increased by appropriately blowing a carbon dioxide gas or an air.

The culture medium used in the cultivation of Spirulina is a liquid culture medium used usually in the cultivation of blue-green algae, for example, a liquid culture medium for blue-green algae, containing 10 to 500 mg/l of phosphorus, 100 to 2000 mg/l of nitrogen, 100 to 2000 mg/l of potassium and a trace amount of metal salts, and preferably a SOT culture medium.

The SOT culture medium is a culture medium containing 16.8 g/l of sodium hydrogencarbonate, 0.5 g/l of dipotassium phosphate, 2.5 g/l of sodium nitrate, 1.0 g/l of potassium sulfate, 7.0 g/l of sodium chloride, 0.2 g/l of magnesium sulfate, 0.04 g/l of calcium chloride, 0.01 g/l of ferrous sulfate, 0.08 g/l of ethylenediaminetetraacetic acid (EDTA), and 1 ml/l of the following A5 solution.

The A5 solution is a solution containing 2.86 g/l of boric acid, 2.50 g/l of manganese chloride, 0.22 g/l of zinc sulfate, 0.08 g/l of copper sulfate, and 0.02 g/l of sodium molybdate.

Spirulina may be industrially prepared by the following method. First, in a laboratory, Spirulina is inoculated in a 0.5 to 5 liter flat flask or Erlenmeyer flask containing a liquid culture medium such as SOT culture medium and then cultivated under a fluorescent lamp of about 10,000 lux at 30 to 40° C. while aerating to prepare a first culture. The resulting first culture is inoculated in a cultivation vessel disposed outside, and then cultivated under sunlight at 30 to 40° C. while ventilating an air to prepare a second culture. The amount of a liquid culture medium used in the preparation of the second culture is usually from 80 to 120 liters. Inoculation of the first culture is usually conducted in a concentration of 20 to 500 mg/l based on the weight of the algal dry biomass of Spirulina. The cultivation time in the preparation of the second culture is usually from 5 to 10 days. The second culture is inoculated in a culture pond outside, and then cultivated under sunlight at 30 to 40° C. while ventilating an air to prepare a third culture. The capacity of the culture pond is usually from 5 to 10 m³. In the cultivation of the third culture, the amount of the second culture to be inoculated and the cultivation time are the same as those in the cultivation of the second culture. As the culture pond, for example, a raceway culture pond with a mixing puddle can be used. In a culture pond having a capacity which is ten times as large as the culture pond in which the third culture was cultivated, the third culture was inoculated as a seed biomass, thereby to attain scale-up of the culture pond. As described above, cultivation is conducted in order in a culture pond having an increased capacity using the culture as a seed biomass and, finally, cultivation is conducted in a culture pond having a capacity of 5000 to 10000 m³.

Spirulina obtained by cultivating according to the above method can be used as it is, or it may be used as a suspension obtained by recovering the cultivated Spirulina with filtering through a filter cloth or paper, washing with water and suspending in water. Furthermore, the cultivated Spirulina may be used as an algal wet biomass obtained by concentrating the culture or suspension, or it may be used as an algal dry biomass obtained by drying the algal wet biomass using a freeze-drying or spray-drying method, or the algal dry biomass may be formed into powders.

Among various zinc compounds used in the present invention, a zinc compound having high safety suited for use in foods is preferable, and examples thereof include inorganic zinc compounds such as zinc chloride, zinc oxide, and zinc sulfate; and organic zinc compounds such as zinc gluconate, zinc stearate, and zinc ascorbate. The zinc compound is preferably a water-soluble zinc compound because it promotes internal absorption upon ingestion of the Spirulina composition or antiallergic food of the present invention. As the water-soluble zinc compound, for example, one or more kinds of water-soluble zinc compounds selected from the group consisting of zinc chloride, zinc sulfate, zinc gluconate and zinc ascorbate can be preferably used.

The zinc compound may be used as it is, or yeast containing the zinc compound incorporated therein during cultivation of the yeast in a culture medium containing the zinc compound can be used as the zinc compound.

The Spirulina composition of the present invention may take any form, and may be a liquid Spirulina composition, a wet Spirulina composition having a moisture content smaller than the liquid Spirulina composition, or a dry Spirulina composition, but is preferably a dry Spirulina composition because of its good utility.

The Spirulina composition of the present invention can be obtained, for example, by mixing Spirulina with a zinc compound. Specific examples of the method of preparing a liquid Spirulina composition include the following methods 1 to 3.

1. To a culture of Spirulina, or a suspension prepared by recovering Spirulina from a culture of Spirulina, washing Spirulina with water and suspending Spirulina again in water and so forth, a water-soluble zinc compound is added, thereby to dissolve the water-soluble zinc compound with mixing.

2. Using a culture medium in which a water-soluble zinc compound is added and dissolved, Spirulina is cultivated and the resulting Spirulina is used as a Spirulina composition of the present invention as it is.

3. Spirulina in a dry state, such as a powder, a water-soluble zinc compound, and water are mixed, thereby dissolving the water-soluble zinc compound in water.

Specific examples of the method of preparing a wet Spirulina composition include the following methods 4 and 5.

4. Moisture is removed from a culture of Spirulina so as to form an algal wet biomass, and then a water-soluble zinc compound is added, thereby dissolving in water contained in the algal wet biomass while mixing.

5. Spirulina in a dry state such as powder, a water-soluble zinc compound and water are mixed in a mixing ratio which enables formation of an algal wet biomass.

Specific examples of the method of preparing a dry Spirulina composition include the following methods 6 to 8.

6. Moisture is removed from a culture of Spirulina so as to form an algal wet biomass, and then a water-soluble zinc compound is added, thereby dissolving in water contained in the algal wet biomass while mixing, followed by drying.

7. Spirulina in a dry state, such as a powder, a water-soluble zinc compound, and water are mixed in a mixing ratio so as to form an algal wet biomass, thereby dissolving the zinc compound in water, followed by drying.

8. Spirulina in a dry state, such as a powder, and a zinc compound are mixed.

The Spirulina composition of the present invention is preferably a Spirulina composition in a dry state and the method of preparing the Spirulina composition in a dry state is preferably the above method 6. The above method 6 will be described in detail below.

First, moisture is removed from a culture of Spirulina so as to form an algal wet biomass. To remove moisture, for example, a concentrator such as filter cloth concentrator or centrifugal concentrator can be used. Moisture is preferably removed until the moisture content in the algal wet biomass is from 70 to 95% by weight, and more preferably from 80 to 90% by weight, because the algal wet biomass and the zinc compound are uniformly mixed with ease and the following drying process can be efficiently conducted.

Then, a zinc compound is added to the resulting algal wet biomass. Although a solid zinc compound or an aqueous solution prepared previously by dissolving the zinc compound in water may be added, the aqueous solution prepared previously by dissolving the zinc compound in water is preferably used. The aqueous solution is preferably used in an amount sufficient to retain an algal wet biomass. The amount of the aqueous solution is preferably from 0.05 to 0.4 parts by volume, and more preferably from 0.1 to 0.3 parts by volume, based on 100 parts by volume of the algal wet biomass.

The zinc compound is added to the algal wet biomass, followed by mixing. Mixing can be conducted by using a mixer such as a kneader or screw type mixer. The mixing time is preferably from 5 to 60 minutes, and more preferably from 10 to 30 minutes, because the algal wet biomass and the zinc compound are well mixed and denaturation of the algal wet biomass is less likely to occur. Mixing is followed by drying.

Drying can be conducted by using a drying machine such as spray-drying machine or freeze-drying machine.

The antiallergic food of the present invention may take any form such as powders, tablets, capsules and granules as long as it contains a Spirulina composition.

The antiallergic food in the form of powders can be prepared, for example, by mixing a dry powder of the Spirulina composition of the present invention, as an essential ingredient, and, optionally, powders of optional ingredients described hereinafter using a powder mixer.

The antiallergic food in the form of tablets can be prepared, for example, by mixing a dry powder of the Spirulina composition of the present invention, as an essential ingredient, and, optionally, powders of optional ingredients described hereinafter using a powder mixer to give a powder mixture, and compressing the powder mixture using a tabletting machine such as rotary tabletting machine. In the preparation of the powder mixture, a wax powder may be optionally added. Examples of the wax powder include powders of magnesium stearate ester, hydrogenated rapeseed oil and sucrose fatty acid ester. The amount of the wax powder is preferably from 0.1 to 10 parts by weight based on 100 parts by weight of the total weight of the dry powder of the Spirulina composition of the present invention and optional ingredients to be optionally added. In the case in which the powder mixture has poor fluidity, the fluidity can be improved by adding a fine silicon dioxide powder to the powder mixture. The amount of the fine silicon dioxide powder is preferably from 0.1 to 2 parts by weight based on 100 parts by weight of the powder mixture.

The antiallergic food in the form of capsules can be prepared, for example, by mixing a dry powder of the Spirulina composition of the present invention, as an essential ingredient, and, optionally, powders of optional ingredients described hereinafter using a powder mixer to yield a powder mixture, and filling gelatin capsules using a capsuling machine. In the preparation of the powder mixture, a wax powder may be optionally added. The amount of the wax powder is preferably the same as in the case of the antiallergic food in the form of tablets. To improve the fluidity, a fine silicon dioxide powder may be added and the amount of the fine silicon dioxide powder is preferably the same in case of the antiallergic food in the form of tablets.

The antiallergic food in the form of granules can be prepared, for example, by mixing a dry powder of the Spirulina composition of the present invention, as an essential ingredient, and, optionally, powders of optional ingredients described hereinafter using a powder mixer to yield a powder mixture, adding water to the powder mixture, mixing them, granulating the mixture using a granulator, and drying the resulting granules with a warm air. The amount of water is preferably from 30 to 100 parts by weight based on 100 parts by weight of the powder mixture. Examples of the granulator include an extruder and fluidized-bed granulator. The temperature of the warm air during drying is usually from 40 to 100° C. Optionally, a wax powder may be added to form a powder mixture. The wax powder may be added in the preparation of the powder mixture, or may be added when the powder mixture is mixed with water. The amount of the wax powder is preferably the same in the case of the antiallergic food in the form of tablets.

Examples of the optional ingredient include vitamins such as vitamin A, vitamin B1, vitamin B2, vitamin B6, vitamin B12, vitamin C, vitamin D, vitamin E, folic acid, pantothenic acid, niacin, and biotin; minerals such as calcium and iron; excipients such as lactose and cellulose; and various perfumes.

The Spirulina composition of the present invention can be added to foods such as bread, udon (wheat noodles), and soba (buckwheat noodles); and beverages such as carbonated water to yield an antiallergic food.

Ingestion of the Spirulina composition and the antiallergic food of the present invention effectively inhibit the production of IL-4, thus preventing or improving allergic diseases. The ingestion amount is preferably from 1 to 10 g per day, and more preferably from 2 to 4 g per day, in terms of the dry weight of the Spirulina composition because it effectively inhibits the production of IL-4 and is suited for ingestion.

### EXAMPLES

The present invention will be described in more detail by the following examples and comparative examples. In the examples and comparative examples, parts and percentages are by weight unless otherwise specified.

### EXAMPLE 1

In a 100 m³ culture pond with a stirrer, containing 20 m³ of a SOT culture medium, Arthrospira (Spirulina) pratensis was inoculated in an amount of 4 kg in terms of the weight of the algal dry biomass and then cultivated under the conditions of an average sunshine duration of 12 hours and an average illuminance of 80,000 lux for 7 days. The culture was filtered through a filter cloth having a sieve opening diameter of 40 μm and Spirulina was concentrated to obtain 163 kg of an algal wet biomass having moisture content of 85%. 300 g of zinc ascorbate (manufactured by Kelatron Corp., Ogden, Utah under the trade name of Zinc Ascorbate Blend 15% Low Lead) was dissolved in 2 liters of water to prepare an aqueous solution of a zinc compound and the total amount of the aqueous solution was added to 163 kg of the algal wet biomass. After the addition, the algal wet biomass was mixed using a kneader for 20 minutes. The algal wet biomass was spray-dried to obtain 23.1 kg of a powdered Spirulina composition. The zinc content of the Spirulina composition was analyzed by an atomic absorption spectrophotometer. As a result, it was 0.11 parts by weight, in terms of zinc, based on 100 parts by weight of the Spirulina composition. 10 Kg of the powdered Spirulina composition was mixed with 0.2 kg of a vitamin C powder, 1 kg of a cellulose powder, 0.1 kg of a fine silicon dioxide powder and 0.1 kg of a magnesium stearate ester powder, and then blue-colored gelatin capsules (size #000) were filled with the resulting powder mixture to prepare capsules. Each of the resulting capsules is referred to as a capsule 1. The amount of the dry Spirulina composition powder in the capsule 1 is 500 mg.

90 healthy volunteers consisting of 60 female adults and 30 male adults were divided at random into the groups 1, 2 and 3; each group consisting of 30 volunteers. After collecting blood from the volunteers belonging to the group 1 using a blood-collecting vessel containing sodium citrate, each volunteer was allowed to continuously ingest 4 capsules 1 per day for 12 weeks. After ingestion, 12 volunteers were further selected from the group 1 and blood was collected using a blood-collecting vessel containing sodium citrate. Using blood (whole blood) collected before and after ingestion of capsules 1 of 12 volunteers, the content of IL-4 in mononucleocytes produced from whole blood was measured.

The measuring method is as follows.

10 ml of whole blood was mixed with 10 ml of a Hank's equilibrium salt (free from calcium chloride, free from magnesium chloride, free from magnesium sulfate, Gibco BRL, Gaithersburg, Md., hereinafter abbreviated to HBSS) to prepare diluted whole blood. The resulting diluted whole blood was subjected to density gradient centrifugation at room temperature under 500 g for 30 minutes using an Accu-Paque gradient reagent (manufactured by Accurate Chemical & Scientific Corp., Westbury, N.Y.) and peripheral blood mononuclear cells (hereinafter abbreviated to PBMC) were collected from the boundary layer. BMC was washed twice with HBSS and the number of the collected PBMC was counted. PBMC was suspended again in a RPMI-1640 culture medium (Gibco BRL) containing a 10% inactivated fetal bovine serum (Atlanta Biologicals, Norcross, Ga., hereinafter abbreviated to FBS) and a 0.1% 50 mg/ml gentamicin solution (Gibco BRL) added therein to obtain a PBMC cell suspension. In the preparation of the PBMC cell suspension, the number of viable cells of PBMC to be stained with tripan blue was controlled to 1×10⁶/ml.

1 ml of the PBMC cell suspension and 20 μg of phytohemagglutinin (hereinafter abbreviated to PHA) were charged in 48-well microplates and IL-4 production of PBMC was stimulated by phytohemagglutinin in a 5% carbon dioxide gas at 37° C. for 72 hours. After 72 hours, the supernatant of the PBMC cell suspension was recovered. The concentration of IL-4 contained in the supernatant was measured and the productivity of IL-4 produced by PBMC due to stimulation of PHA was evaluated. The results are shown in Table 1. The measurement results indicate an average value of the measured values of 12 volunteers (this is repeated in the following). The lower the concentration of IL-4, the more the production of IL-4 is inhibited. In the measurement, High Sensitivity Quantikine Human IL-4 ELISA kit (R&D Systems, Minneapolis, Minn.) having a detection limit of 0.25 pg/ml was used. As is apparent from the results, the production of IL-4 is inhibited by ingestion of the Spirulina composition of the present invention.

### EXAMPLE 2

5 kg of a dry Spirulina composition powder was mixed with 0.1 kg of a vitamin C powder, 5.5 kg of a cellulose powder, 0.1 kg of a fine silicon dioxide powder and 0.1 kg of a magnesium stearate ester powder, and then blue-colored gelatin capsules (size #000) were filled with the resulting powder mixture to prepare capsules. Each of the resulting capsules is referred to as capsule 2. The amount of the dry Spirulina composition powder in the capsule 2 is 250 mg. Using the volunteer group of the group 2, the IL-4 production inhibitory effect of the capsule 2 was evaluated in the same manner as in Example 1. The measurement results are shown in Table 1. As is apparent from the results, the production of IL-4 is inhibited by ingestion of the Spirulina composition of the present invention.

### COMPARATIVE EXAMPLE 1

10 kg of a cellulose powder, 0.1 kg of a fine silicon dioxide powder and 0.1 kg of a magnesium stearate ester powder were mixed, and then blue-colored gelatin capsules (size #000) were filled with the resulting powder mixture to prepare capsules. Each of the resulting capsules is referred to as a capsule 3. The amount of the dry Spirulina composition powder in the capsule 2 is 0 mg. Using the volunteer group of the group 3, the IL-4 production inhibitory effect of the capsule 3 was evaluated in the same manner as in Example 1. The measurement results are shown in Table 1. As is apparent from the results, the production of IL-4 is not inhibited when the Spirulina composition of the present invention is not ingested.

TABLE 1

| | | | Comparative |
|---|---|---|---|
| | Example 1 | Example 2 | Example 1 |
| | Capsule 1 | Capsule 2 | Capsule 3 |
| IL-4 productivity (pg/ml) | 21.9 | 24.2 | 20.5 |
| before ingestion of capsules | | | |
| IL-4 productivity (pg/ml) | 14.8 | 21.5 | 19.8 |
| after ingestion of capsules | | | |

### EXAMPLE 3

430 mg of the Spirulina composition prepared in Example 1 was added to 50 ml of a phosphate buffered saline (GibcoBRL, hereinafter abbreviated to PBS), and then the mixture was vigorously shaken and centrifuged, thereby to recover the supernatant. The supernatant was filtered through a membrane filter having a pore diameter of 0.45 μm, and was further filtered through a membrane filter having a pore diameter of 0.2 μm to obtain a filtrate. The resulting filtrate is referred to as a Spirulina composition filtrate.

Whole blood was collected from 6 healthy volunteers using a blood-collecting vessel containing sodium citrate. The PBMC cell suspension was prepared in the same manner as in Example 1, except that the number of viable cells of PBMC to be stained with tripan blue was controlled to 2×10⁶/ml.

250 μl of the PBMC cell suspension, 125 μl of the Spirulina composition filtrate, 125 μl of a RPMI-1640 culture medium containing 10% FBS added therein, and 10 μg of PHA were charged in 48-well microplates and IL-4 production of PBMC was stimulated by phytohemagglutinin in a 5% carbon dioxide gas at 37° C. for 72 hours. After 72 hours, the supernatant of the PBMC cell suspension was recovered.

The concentration of IL-4 contained in the supernatant was measured in the same manner as in Example 1 and the productivity of IL-4 produced by PBMC due to stimulation of PHA was evaluated. The results are shown in Table 2. The measurement results indicate an average value of the measured values of 6 volunteers (this is repeated in the following

### COMPARATIVE EXAMPLE 2

In the same manner as in Example 3, except that a commercially available spray-dried powder (Earthrise Nutritionals, Petaluma, Calif.) of Arthrospira (Spirulina) pratensis was used in place of the Spirulina composition prepared in Example 1, a filtrate was obtained. This filtrate is called the comparative Spirulina filtrate. In the same manner as in Example 3, except that this comparative Spirulina filtrate was used, the productivity of IL-4 produced by PBMC was evaluated. The results are shown in Table 2. The zinc content of the spray-dried powder of Arthrospira (Spirulina) pratensis was analyzed by an atomic absorption spectrophotometer. As a result, it was 0.0015 parts by weight based on 100 parts by weight of the dry powder.

The productivity of IL-4 of Example 3 is lower than that of Comparative Example 2. As is apparent from the results, the production of IL-4 of the Spirulina composition of the present invention is inhibited by a conventional commercially available Spirulina having law zinc content.

TABLE 2

| | Example 3 | Comparative |
|---|---|---|
| | Spirulina | Example 1 |
| | composition | Spirulina |
| IL-4 productivity | 27.0 | 37.9 |
| (pg/ml) | | |

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A Spirulina composition comprising Spirulina and a zinc compound, wherein the content of the zinc compound is from 0.04 to 5 parts by weight, in terms of zinc, based on 100 parts by weight of the dry weight of Spirulina.

2. The Spirulina composition according to claim 1, wherein the zinc compound is a water-soluble zinc compound.

3. The Spirulina composition according to claim 2, wherein the water-soluble zinc compound is at least one water-soluble zinc compound selected from the group consisting of zinc chloride, zinc sulfate, zinc gluconate and zinc ascorbate.

4. The Spirulina composition according to claim 1, which contain the zinc compound in an amount of 0.07 to 0.5 parts by weight, in terms of zinc, based on 100 parts by weight of the dry weight of Spirulina.

5. An antiallergic food comprising the Spirulina composition of claim 1.

6. A method for prevention or improvement of allergic diseases, which includes ingestion of the Spirulina composition of claim 1.

7. The Spirulina composition according to claim 2, which contain the zinc compound in an amount of 0.07 to 0.5 parts by weight, in terms of zinc, based on 100 parts by weight of the dry weight of Spirulina.

8. The Spirulina composition according to claim 3, which contain the zinc compound in an amount of 0.07 to 0.5 parts by weight, in terms of zinc, based on 100 parts by weight of the dry weight of Spirulina.
